(19) United States
(12) Patent Application Publication
(10) Pub. No.: US 2023/0039200 A1
(43) Pub. Date: Feb. 9, 2023

(54) LASER ADDITIVE MANUFACTURING METHOD FOR PRODUCING POROUS LAYERS

(71) Applicant: THE REGENTS OF THE UNIVERSITY OF CALIFORNIA, Oakland, CA (US)

(72) Inventors: Andrea Perego, Oakland, CA (US); Iryna Zenyuk, Oakland, CA (US); Yoonjin Won, Oakland, CA (US)

(73) Assignee: THE REGENTS OF THE UNIVERSITY OF CALIFORNIA, Oakland, CA (US)

(21) Appl. No.: 17/790,022

(22) PCT Filed: Dec. 30, 2020
(86) PCT No.: PCT/US2020/067531

§ 371 Date: Jun. 29, 2022

Related U.S. Application Data
(60) Provisional application No. 62/955,704, filed on Dec. 31, 2019.

(51) Int. Cl. B22F 3/11; B33Y 10/00; B33Y 70/00; A61L 27/56; A61L 27/04

(52) U.S. Cl. CPC B22F 3/1134; B33Y 10/00; B33Y 70/00; B22F 3/1109; A61L 27/56; A61L 27/04; B22F 2999/00

(57) ABSTRACT

Provided herein are manufacturing methods, e.g., comprising: (1a) forming a layer, including: depositing a starting material including a mixture of a metal and a sacrificial material; and applying a laser beam to the deposited starting material to consolidate the deposited starting material and form the layer; (1b) optionally repeating (1a) one or more times; and (1c) at least partially removing the sacrificial material to form a porous metal part.

### CROSS-REFERENCE TO RELATED APPLICATIONS

This application claims the benefit of U.S. Provisional Application No. 62/955,704, filed Dec. 31, 2019, the content of which is incorporated herein by reference in its entirety.

### Background

In traditional metal three-dimensional (3D) printing, and in particular in selective laser melting (SLM), a solid part is formed layer-by-layer from a metal powder by using a laser that selectively melts a material of each layer. Ordered porous parts can be formed and tuned by giving a printing machine a precisely constructed mesh as an input. The resolution of a mesh and therefore parameters of a porous part (e.g., porosity, pore size distribution, and surface area) are constrained by a spot size of a laser used, and the interaction between a material and a laser beam, namely the thermal conductivity of the material. The typical spot size of lasers are in the range of tens to hundreds of microns. For applications that specify a porosity in the micron or sub-micron range, a precise control of porosity is not achievable using a laser pattern as the sole degree of freedom that can be changed. Different solutions should be adopted to decrease a pore size in a material.

It is against this background that a need arose to develop the embodiments described herein.

### SUMMARY

Embodiments of this disclosure are directed to a laser additive manufacturing method of forming porous metal parts with hierarchical tunable porosity at different length scales.

The method is implemented in certain embodiments by adding a salt powder (e.g., sodium chloride (NaCl)) as a sacrificial template in a metal powder used in SLM technique to form pores in a solid part. The sacrificial template is etched or otherwise removed at the end of the method, forming a pore network in the solid part.

A salt powder is used in certain embodiments to facilitate the etching procedure, which does not involve toxic solvents (e.g., hydrofluoric acid (HF) for silica-based hard templates) and also does not introduce undesired modifications in a metal part.

Some embodiments include a manufacturing method comprising: (1a) forming a layer, including: depositing a starting material including a mixture of a metal and a sacrificial material; and applying a laser beam to the deposited starting material to consolidate the deposited starting material and form the layer; (1b) optionally repeating (1a) one or more times; and (1c) at least partially removing the sacrificial material to form a porous metal part. In some embodiments, depositing the starting material includes depositing the starting material as a powder. In some embodiments, the starting material is a mixture of a power of the metal and a powder of the sacrificial material. In some embodiments, the powder of the sacrificial material includes particles having an average size in a range of about 1 nm to about 70 μm, about 1 nm to about 50 μm, about 1 nm to about 10 μm, about 1 nm to about 1μm, about 1 nm to about 800 nm, or about 1 nm to about 500 nm. In some embodiments, the powder of the sacrificial material includes particles having a size distribution that is monodisperse. In some embodiments, the powder of the sacrificial material includes particles having a size distribution that is polydisperse. In some embodiments, the resulting porous metal part includes pores having an average size and a size distribution corresponding to an average size and a size distribution of particles of the sacrificial material. In some embodiments, the resulting porous metal part has a porosity corresponding to a ratio of the powder of the sacrificial material and the power of the metal. In some embodiments, applying the laser beam to the deposited starting material is according to a mesh pattern. In some embodiments, the resulting porous metal part includes additional pores having an average size and a size distribution corresponding to the mesh pattern. In some embodiments, the sacrificial material remains in a liquid or solid state while applying the laser beam to the deposited starting material. In some embodiments, the starting material is an ionic salt. In some embodiments, removing the sacrificial material includes dissolving the sacrificial material in a solvent. In some embodiments, dissolving the sacrificial material is performed at an elevated temperature.

Additional embodiments include a manufacturing method comprising: (2a) forming a first layer, including: depositing a first starting material including a first mixture of a metal and a sacrificial material; and applying a laser beam to the deposited first starting material to consolidate the deposited first starting material and form the first layer; (2b) optionally repeating (2a) one or more times; (2c) forming a second layer on the first layer, including: depositing a second starting material including a second mixture of the metal and the sacrificial material; and applying a laser beam to the deposited second starting material to consolidate the deposited second starting material and form the second layer; (2d) optionally repeating (2c) one or more times; and (2e) at least partially removing the sacrificial material to form a porous metal part. In some embodiments, depositing the first starting material includes depositing the first starting material as the first mixture of a power of the metal and a powder of the sacrificial material, and depositing the second starting material includes depositing the second starting material as the second mixture of a power of the metal and a powder of the sacrificial material. In some embodiments, an average size or a size distribution of particles of the sacrificial material in the first mixture is different than an average size or a size distribution of particles of the sacrificial material in the second mixture. In some embodiments, a ratio of the powder of the sacrificial material and the power of the metal in the first mixture is different than a ratio of the powder of the sacrificial material and the power of the metal in the second mixture. In some embodiments, applying the laser beam to the deposited first starting material is according to a first mesh pattern. In some embodiments, the resulting porous metal part includes additional pores having an average size and a size distribution corresponding to the first mesh pattern. In some embodiments, applying the laser beam to the deposited second starting material is according to a second mesh pattern. In some embodiments, the resulting porous metal part includes additional pores having an average size and a size distribution corresponding to the second mesh pattern. In some embodiments, the first mesh pattern is different than the second mesh pattern.

Additional embodiments include a porous metal part formed by the manufacturing methods of any of the above embodiments. In some embodiments, the porous metal part is a catalyst support. In some embodiments, the porous metal part is a porous transport layer of a hydrogen generator. In some embodiments, the porous metal part is a porous medium of a heat pipe. In some embodiments, the porous metal part is a component of an implantable device.

### BRIEF DESCRIPTION OF THE DRAWINGS

FIG. 1 shows an embodiment of a schematic of the SLS process with the controlling parameters.

FIG. 2 shows an embodiment of result for SLM of a stainless-steel part.

FIG. 3(a) shows an embodiment of an optical image of cross section of a pure metal layer, laser power: 100 W. FIG. 3(b) shows an embodiment of an optical image of a metal-pore former composite, 50% volume ratio. FIG. 3(c) shows an embodiment of an SEM image of metal-pore former composite, 50% volume ratio.

### DETAILED DESCRIPTION

In some embodiments according to a first aspect, a manufacturing method includes: (1a) forming a layer, including: depositing a starting material including a mixture of a metal and a sacrificial material; and applying a laser beam to the deposited starting material to consolidate the deposited starting material and form the layer; (1b) optionally repeating (1a) one or more times; and (1c) at least partially removing the sacrificial material to form a porous metal part.

In some embodiments of the manufacturing method according to the first aspect, depositing the starting material includes depositing the starting material as a powder. In some embodiments, the starting material is a mixture of a power of the metal and a powder of the sacrificial material. In some embodiments, the powder of the sacrificial material includes particles having an average size in a range of about 1 nm to about 70 μm, about 1 nm to about 50 μm, about 1 nm to about 10 μm, about 1 nm to about 1 μm, about 1 nm to about 800 nm, or about 1 nm to about 500 nm. In some embodiments, the powder of the sacrificial material includes particles having a size distribution that is monodisperse (e.g., having a standard deviation of about 30% or less, about 20% or less, or about 10% or less, relative to the average size). In some embodiments, the powder of the sacrificial material includes particles having a size distribution that is polydisperse (e.g., having a standard deviation of greater than 30%, relative to the average size). In some embodiments, the resulting porous metal part includes pores having an average size and a size distribution corresponding to an average size and a size distribution of particles of the sacrificial material. In some embodiments, the resulting porous metal part has a porosity corresponding to a ratio (e.g., by volume) of the powder of the sacrificial material and the power of the metal. In some embodiments, the resulting porous metal part has a porosity is greater than about 60% (e.g., 60%, 65%, 70%, 75%, 80%, 85%, or more). In embodiments, the amount of metal in the mixture of a metal and a sacrificial material is about 25, 30, 35, 40, 45, 50, 55, 60, 65, 70, 75 vol.% with the remaining vol.% being sacrificial material.

In some embodiments of the manufacturing method according to the first aspect, the metal is one or more transition metal, post-transition metal, or alloy. In some embodiments, the metal is steel (e.g., stainless steel). The metal in some embodiments is at least one of aluminum (Al), scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), indium (In), tin (Sn), lanthanum (La), hafnium (Hf), tantalum (Ta), tungsten (W), rhenium (Re), osmium (Os), iridium (Ir), platinum (Pt), and gold (Au). In some embodiments, the metal is at least one of titanium, copper, aluminum, and nickel.

In some embodiments of the manufacturing method according to the first aspect, the sacrificial material is an ionic salt, such as an alkali metal salt or an alkaline earth metal salt. Specific examples include NaCl, KCl, and the like. In some embodiments, the sacrificial material is a metal oxide, such as aluminum oxide. In some embodiments, the sacrificial material is a silicon oxide. In some embodiments, the sacrificial material is non-toxic and/or soluble in an aqueous solvent (e.g., water).

In some embodiments of the manufacturing method according to the first aspect, applying the laser beam to the deposited starting material is according to a mesh pattern. In some embodiments, the resulting porous metal part includes additional pores having an average size and a size distribution corresponding to the mesh pattern.

In some embodiments of the manufacturing method according to the first aspect, the sacrificial material remains in a liquid or solid state while applying the laser beam to the deposited starting material. In some embodiments, the starting material is an ionic salt. In some embodiments, the ionic salt is sodium chloride. In some embodiments, removing the sacrificial material includes dissolving the sacrificial material in a solvent. In some embodiments, the solvent is water. In some embodiments, dissolving the sacrificial material is performed at an elevated temperature (e.g., above 25° C. and up to about 100° C. or greater).

In some embodiments of the manufacturing method according to the first aspect, further surface treatment is performed on the porous metal. For example, a thermal treatment may be performed, such as surface oxidation (e.g., through either annealing in oxidant atmosphere or with chemical attack) and surface nitridation (e.g., treated with ammonia at high temperature (900° C.) to form nitride on the surface, e.g., Ti nitride). In some embodiments, an electrochemical treatment may be performed, such as electroplating (e.g., deposition of a metal coating on top of the structure using an electrochemical cell with metal ions dissolved in the electrolyte) or a change in the composition of stainless steel (e.g., chromium enrichment performed with an electrochemical process that removes iron from the steel, increasing the chromium concentration at the surface).

In additional embodiments according to a second aspect, a manufacturing method includes: (2a) forming a first layer, including: depositing a first starting material including a first mixture of a metal and a sacrificial material; and applying a laser beam to the deposited first starting material to consolidate the deposited first starting material and form the first layer; (2b) optionally repeating (2a) one or more times; (2c) forming a second layer on the first layer, including: depositing a second starting material including a second mixture of the metal and the sacrificial material; and applying a laser beam to the deposited second starting material to consolidate the deposited second starting material and form the second layer; (2d) optionally repeating (2c) one or more times; and (2e) at least partially removing the sacrificial material to form a porous metal part.

In some embodiments of the manufacturing method according to the second aspect, depositing the first starting material includes depositing the first starting material as the first mixture of a power of the metal and a powder of the sacrificial material, and depositing the second starting material includes depositing the second starting material as the second mixture of a power of the metal and a powder of the sacrificial material. In some embodiments, an average size or a size distribution of particles of the sacrificial material in the first mixture is different than an average size or a size distribution of particles of the sacrificial material in the second mixture. In some embodiments, a ratio (e.g., by volume) of the powder of the sacrificial material and the power of the metal in the first mixture is different than a ratio (e.g., by volume) of the powder of the sacrificial material and the power of the metal in the second mixture. In some embodiments, the resulting porous metal part has a porosity corresponding to a ratio (e.g., by volume) of the powder of the sacrificial material and the power of the metal. In some embodiments, the resulting porous metal part has a porosity is greater than about 60% (e.g., 60%, 65%, 70%, 75%, 80%, 85%, or more). In embodiments, the amount of metal in the mixture of a metal and a sacrificial material is about 25, 30, 35, 40, 45, 50, 55, 60, 65, 70, 75 vol.% with the remaining vol.% being sacrificial material.

In some embodiments of the manufacturing method according to the second aspect, the metal is one or more transition metal, post-transition metal, or alloy. In some embodiments, the metal is steel (e.g., stainless steel). The metal in some embodiments is at least one of aluminum (Al), scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), indium (In), tin (Sn), lanthanum (La), hafnium (Hf), tantalum (Ta), tungsten (W), rhenium (Re), osmium (Os), iridium (Ir), platinum (Pt), and gold (Au). In some embodiments, the metal is at least one of titanium, copper, aluminum, and nickel.

In some embodiments of the manufacturing method according to the second aspect, the sacrificial material is an ionic salt, such as an alkali metal salt or an alkaline earth metal salt. Specific examples include NaCl, KCl, and the like. In some embodiments, the sacrificial material is a metal oxide, such as aluminum oxide. In some embodiments, the sacrificial material is a silicon oxide. In some embodiments, the sacrificial material is non-toxic and/or soluble in an aqueous solvent (e.g., water).

In some embodiments of the manufacturing method according to the second aspect, applying the laser beam to the deposited first starting material is according to a first mesh pattern. In some embodiments, the resulting porous metal part includes additional pores having an average size and a size distribution corresponding to the first mesh pattern. In some embodiments of the manufacturing method according to the second aspect, applying the laser beam to the deposited second starting material is according to a second mesh pattern. In some embodiments, the resulting porous metal part includes additional pores having an average size and a size distribution corresponding to the second mesh pattern. In some embodiments, the first mesh pattern is different than the second mesh pattern.

In some embodiments of the manufacturing method according to the second aspect, the sacrificial material remains in a liquid or solid state while applying the laser beam to the deposited first starting material and while applying the laser beam to the deposited second starting material. In some embodiments, the starting material is an ionic salt. In some embodiments, the ionic salt is sodium chloride. In some embodiments, removing the sacrificial material includes dissolving the sacrificial material in a solvent. In some embodiments, the solvent is water. In some embodiments, dissolving the sacrificial material is performed at an elevated temperature (e.g., above 25° C. and up to about 100° C. or greater).

In some embodiments of the manufacturing method according to the second aspect, further surface treatment is performed on the porous metal. For example, a thermal treatment may be performed, such as surface oxidation (e.g., through either annealing in oxidant atmosphere or with chemical attack) and surface nitridation (e.g., treated with ammonia at high temperature (900° C.) to form nitride on the surface, e.g., Ti nitride). In some embodiments, an electrochemical treatment may be performed, such as electroplating (e.g., deposition of a metal coating on top of the structure using an electrochemical cell with metal ions dissolved in the electrolyte) or a change in the composition of stainless steel (e.g., chromium enrichment performed with an electrochemical process that removes iron from the steel, increasing the chromium concentration at the surface).

Further embodiments are directed to the porous metal part formed by the manufacturing methods of the foregoing embodiments. In some embodiments, the porous metal part is a catalyst support. In some embodiments, the porous metal part is a porous transport layer of a hydrogen generator. In some embodiments, the porous metal part is a porous medium of a heat pipe. In some embodiments, the porous metal part is a component of an implantable device.

Certain embodiments of this disclosure leverage the use of a sacrificial template coupled with selective laser melting (SLM) technique. The sacrificial template in certain embodiments is introduced as an additive to a metal powder used as a starting material in an SLM printing machine. It is applicable to commercial machines and does not require any modifications to SLM configurations.

A correct particle size of a sacrificial template's powder can be obtained from a chemical manufacturer based on specifications, or controlled using techniques such as ball milling. In some embodiments, the size and a ratio between a metal and a sacrificial material in a mixture will determine the porosity and the pore size distribution of the final part. Those parameters can be optimized considering criteria of porosity coupled with a mechanical resistance of the final part that will decrease.

By choosing a correct salt (based on melting and boiling points) in the mixture, and by tuning the laser power accordingly, it can be ensured that both the salt and the metal stay in the solid or liquid phase during the manufacturing process—in this way, the salt is embedded in the metal and can be etched at a subsequent time exploiting its solubility by boiling the part in de-ionized (DI) water.

TABLE 1
Table indicating the boiling and the melting points of various metals
compared to the specific enthalpy for the phase transitions.

| | Units | SS316L | Cu | Al | Ti | NaCl |
|---|---|---|---|---|---|---|
| Melting point | K | 1648 | 1358 | 934 | 1941 | 1073 |
| (K) | | | | | | |
| Boiling Point | K | — | 2835 | 2740 | 3560 | 1686 |
| (K) | | | | | | |
| ΔH Ambient- | kJ*(kg)⁻¹ | 662.0 | 413.4 | 578.3 | 887.3 | 669.7 |
| Fusion | | | | | | |
| ΔH Fusion | kJ*(kg)⁻¹ | 285.0 | 205.4 | 399.9 | 295.6 | 478.3 |
| ΔH Fusion- | kJ*(kg)⁻¹ | — | — | — | — | 659.6 |
| boiling | | | | | | |
| Total Enthalpy | kJ*(kg)⁻¹ | 947.0 | 618.8 | 978.2 | 1182.9 | 1807.6 |

Table 1 shows the energetics and the phase transition temperatures for some materials of interest in SLM applications. Given that a correct laser power is set on an SLM machine, the choice of NaCl as a templating additive ensures that for a selected metal, when the metal is completely fused the salt is also in the solid or liquid phase in the part, and therefore it can be etched afterwards. Moreover, a laser interaction with NaCl, depending on the wavelength of the laser and the absorption coefficient, may result in an ineffective energy transfer, leaving the salt powder in the material without changing its phase.

An etching process, giving the nature of the sacrificial template, can be performed in DI water and can be enhanced by increasing the temperature. The efficiency of the etching is dependent on the size and the nature of pores. It can be hindered by the presence of capillary pores that can impede water from etching a salt, or salt zones covered entirely by a metal which impedes access of a liquid. This can be mitigated by increasing the salt to metal ratio in an initial mixture to create percolation pathways that facilitate the etching process.

An application of the SLM technique involves the fabrication of a precisely tuned porous part with computer-aided design (CAD)-drawn mesh patterns. A pore size is constrained by a spot size of a laser used in an SLM machine, as well as the interaction of the laser with a material. Typical values are in the range of hundreds of microns.

In some embodiments, in the method that is disclosed, a further, stochastic level of control of porosity is added, where a particle size of a sacrificial template is determining a magnitude of dimensions of pores, unlocking the possibility to reach down to the nanometer scale.

Possible applications for resulting porous parts can be found in the manufacturing of well-tailored electrodes for electrochemical systems such as electrolyzers, where a compromise between large size porosity for favoring the mass transport has to be weighted with having large surface area for improving a reaction rate.

Moreover, exploiting the solubility of a salt in water for the etching process is intrinsically safer and less harmful for the environment, compared to sacrificial templates involving removal via wet chemistry such as silica, which has to be treated with hydrofluoric acid to obtain hollow structures.

Stages for the development of certain embodiments of the method can involve:
(1): A proof of concept can be fabricated with a spherical silica gel hard template and stainless steel: silica gel spheres can be mixed with stainless steel powder in a ball milling setup before being transferred to an SLM machine. A rectangular solid film can be fabricated and scanning electron microscopy (SEM) analysis can be performed to assess the distribution of the sacrificial material in the resulting part. No etching will be performed at this stage.
(2): Stainless steel can be mixed with sodium chloride and transferred to an SLM machine. A rectangular solid film can be fabricated and etching can be performed by submerging the film in boiling water. SEM-energy-dispersive X-ray spectroscopy (SEM-EDS) analysis can be performed for assessment of the porosity and the presence of residual salt in the part.
(3) Optimization: Different salt powder sizes, different laser power and different metal to salt ratio can be evaluated with a sensitivity analysis to control properties of a porous part. Porosimetry and nitrogen/krypton physisorption methods can be employed to determine properties of the part (e.g., surface area and pore size distribution).
(4) Layers with hierarchical porosity: The process can be coupled with 3D meshes to have a porous layer with two different degrees of porosity, with a first determined by a mesh pattern drawn by a machine (in the micrometer scale) and a second, in the nanometer scale, by a sacrificial template.
(5) Porosity gradient: As a further stage, a porosity gradient can be introduced by mesh developing and by tuning a local composition of a mixture.

Example applications include:
Use of titanium supports to grow body tissue: 3D printing combined with porosity allow porous parts to fit in various locations in the human body.
Biocompatible supports for spine surgery.
Porous titanium sheets as a catalyst support and a porous transport layer in hydrogen generators.

### Additional Information and Embodiments

### Objectives

Some embodiments include an alternative route for the production of a class of porous media, and their application in the energy field. In the present framework, with consideration of green energy and environmental compatibility, a fabrication process should focus on cost and production volume, but also take into account environmental implications, such as compatibility with renewable sources, the sustainability of materials involved and the production of toxic wastes.

### Porous Media

Porous materials are used as active surfaces in certain embodiments that involve energy transport through a solid and energy exchange across a large solid-fluid interfacial surface area. The rational design of porous media is often employed to create combinations of electrical, thermal, and fluidic transport. Transport characteristics of well-ordered porous media are governed by pore distribution, porosity, and morphology. The combination of improved transport physics can support performance breakthroughs in applications ranging from electrode-electrolyte interfaces in electrochemical devices, to capillary-fed heat pipes, and vapor chambers.

### Porous Media Criteria—Why Hierarchical or Heterogeneous Media

In many applications, the coupled criteria of mass and heat transport often result in trade-offs with respect to morphological variables of porous media (e.g., porosity, pore sizes, and surface roughness). For example, liquid transport within porous media in capillary-fed heat pipes or vapor chambers competes against viscous resistance associated with small pores. Thermal conductivity generally decreases with porosity while hydraulic permeability increases.

In electrochemical devices such as electrolyzers, heterogeneous reactions occur at an electrode/electrolyte interface: therefore, the design of the optimal configuration should manage a trade-off between transport resistances of reactants/products, related to the formation of bubbles in a liquid phase by the electrochemical reaction and the rate of the reaction itself that is related to the specific surface area. Large pore size can enhance the transport properties of the electrode, enhancing bubble removal, but at the same time the surface area available for the reaction is decreased. A hierarchical structure for the electrode can grant both the large-scale porosity for bubble removal and an additional smaller scale porosity for the reaction to occur at a high rate.

### Process of Certain Embodiments

The microstructure of comparative porous media typically has been amorphous, meaning that a pore distribution is irregular without well-defined unit cell. Such materials exhibit a single set of effective properties that are uniform but with large deviations throughout an entire volume. The trade-offs indicate the use of both heterogeneous porous structures to optimize for the spatially segregated dominant transport processes at different locations in a thermofluidic device and hierarchical morphology to account for different length scales associated with each transport phenomenon. Therefore, some embodiments include a manufacturing method in order to prepare heterogeneous, multi-scale porous media with hierarchy to push toward the limits of thermofluidic devices. The use of spatially varying materials can help to simultaneously optimize the thermal conductivity near the source, capillary-driven liquid flow into a heated region, and interfacial heat and mass transfer within a porous volume. The methodologies can include (1) selective laser sintering with sacrificial particles and follow up surface treatment, (2) thermofluidic characterizations with precise X-ray topography analysis, and (3) machine learning-based design of manufacturing parameters and morphological parameters. These multiphase transport physics aspects can lead to the creation of next-generation thermal management devices for spacecraft systems.

### Technology

Hierarchical or heterogeneous materials in certain embodiments can be demonstrated by using nanotechnologies including particle sintering method. However, such methods generally fail to provide porous media at a large-scale.

Another way is to vary power density, hatch distance, pulse duration, and so forth during a laser sintering technique, which can be either expensive or lead to undefined porosity.

For defined porosity, a 3D CAD pattern can be created but this process is constrained by a laser spot size (about 70 μm).

Selective laser sintering (SLS) is an additive manufacturing (AM) method for fabricating sintered parts from plastic, metal, ceramic or polymer. In in certain embodiments of this AM method a high power laser raster-scans a powder layer-by-layer to produce a pre-programmed topologically-complex 3D object. In certain embodiments, sintering involves a process where particles are joined under heat without undergoing melting. The degree of sintering depends on the laser peak power and hence pulsed control is typically used. Selective laser melting (SLM) or direct laser melting (DLM) is different from the SLS because the process involves melting and fusing metal powders. Typically, in the SLM process, porosity is undesirable as metal parts that are porous have lower strength and can be permeable to liquid and gas, which is undesirable for manufacturing solid parts. Undesired porosity in the solid parts is attributed to inadequate fusion or melting, over-melting and overheating, and gas confinement during melting. Processing parameters and powder size and shape are parameters for porosity formation in a layer during the DLM. One way to achieve porosity is to mix spherical and non-spherical powders in the DLM process; however, the process is empirical and does not allow for a precise control. Furthermore, the achieved porosity is generally geometrically undefined.

Several metrics are in place to relate to manufacturing parameters. Volume energy density is one of these metrics, which can be used to understand how various parameters control an output energy, where a lower energy will result in higher porosity in DLM and also in the SLS:

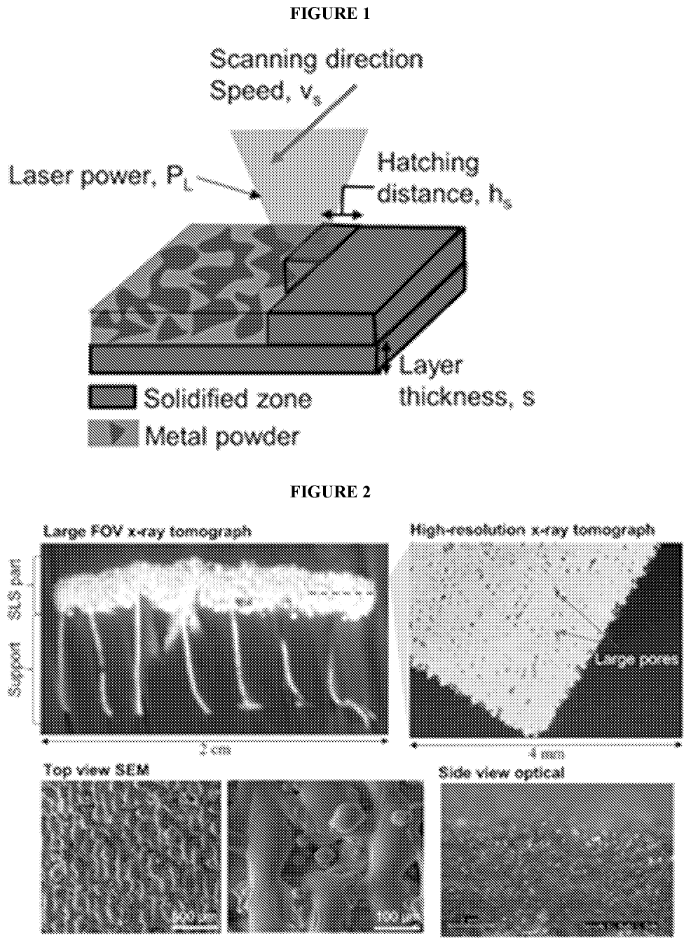

E=P_{L}/(vₛhₛs)

where P_{L} is the power of a laser, vₛ is the speed of a scan, hₛ is the hatching distance and s is the layer thickness. FIG. 1 shows a schematic of the SLS process with the controlling parameters. Porosity is inversely proportional to energy density. The highest porosity is shown to be reached with the variation of the hatching distance, where a sufficiently large hatching distance can represent geometrically defined porosity. Geometrically defined porosity is based on lattice structures, where pore sizes on the order of 100 μm can be achieved.

SLM is identified as an ideal process since it meets two criteria: being driven by a laser, the energy input of the process is in the form of electrical energy, which can be compatible with renewable energy from the grid. Also, it uses metal powders as a raw material, which can derive from recycling metal from an electronic industry.

SLM technique can be used to produce metal parts with rather complex geometry, and porous layers fabricated by SLM technique are desired in the biomedical field, in particular for the production of prosthetic supports for joints and bones. The material mostly used is titanium for its biocompatibility, and SLM is an efficient way to process the material.

Porosity is obtained in certain embodiments by providing a printing machine a specific geometric input to tailor a trade-off between porosity and mechanical properties. This also allows the possibility to introduce a gradient in porosity by correctly introducing a thickness dependency in parameters of a particular unit cell, e.g., gyroid-like.

Some applications of SLM can be found in the fabrication of tailored porous electrodes for the production of hydrogen, namely as electrodes for oxygen evolution reaction. In that particular case, the performances are heavily dependent on the management of the oxygen bubbles generated in the electrochemical reaction so a well-engineered porous structure is desired to remove the products from the reaction sites, thereby increasing the turnover of reactants.

Embodiments of Advanced Manufacturing Methodology: Selective Laser Melting with Sacrificial Pore Former

In some embodiments, the fabrication of porous layers by SLM, focusing on the achievement of control over the morphology, the surface area of a porous layer as well as the pore size at different length scales, from millimeter to tens of nanometers. To achieve control over such small dimensions, in certain embodiments, departure is made from the comparative approach that is constrained by laser/metal interaction and an improved approach is developed to address the problem.

While comparative SLM approach sets a particular geometry through a characteristic equation that is given as an input to a printing machine, a sacrificial template method is leveraged in certain embodiments of the disclosed approach. In this stochastic approach, a pore former is added to an initial material and it is etched after formation, leaving a void space inside a bulk when removed. Sacrificial pore formers are broadly used to introduce porosity to materials with hierarchical porosity.

In certain embodiments of the disclosed approach, a length scale of pores does not depend on a laser-metal interaction but instead on dimensions of particles of a templating additive, which can be monodispersed and also with a certain degree of hierarchy, opening a massive set of possibilities to choose the best configuration for a vast range of different applications.

The general features for a sacrificial pore former material in certain embodiments are inertness in the environment, controllable shape and size and the possibility to etch the material after the process. Silica is a pore former generally employed in wet processes to obtain ordered pore structures, often used in catalysis due to their high surface area. Silica is stable and it can be shaped with good control in dimensions. Its main drawback is in the etching process, which involves treatment in hydrofluoric acid. This implies several environmental and safety concerns, and special treatment and precautions to be adopted when using that type of substance.

In certain embodiments, the SLM technique does not rely on wet chemical processes, and therefore possible pore formers can be extended to other substances that are not compatible with solvents. One possible class of substances that can be employed in certain embodiments of the process is ionic salts. They have a fairly high boiling and melting point (see Table 1) and they can be grinded down to the desired particle size. The main advantage is they can be etched by dissolving in water, and the process is greatly enhanced with the temperature. By omitting acid/alkaline solvents for the etching, a metallic surface of a part is not affected.

To be compatible with SLM, in certain embodiments, a salt should meet some criteria: first it should remain in the solid or liquid state throughout a melting process: in Table 1, the energetics involved in the phase transitions are reported. Sodium chloride (NaCl) is compatible with various metals of interest, and also it is inexpensive and readily available. Also it has high solubility in water, which is instrumental to the etching phase: no hazardous chemical is involved, making it safe and environmental friendly.

The energy involved to vaporize sodium chloride is greater than the energy involved to liquefy a metal of interest in certain embodiments. Therefore, tuning the correct laser power for melting the metal, NaCl salt will stay in the bulk material throughout the process either in the solid or liquid phase while the metal is melting. On top of that, the energy effectively transferred to the salt is also dependent on interaction between sodium chloride and the laser pulse, with dependency given by the absorption coefficient at the specific laser wavelength. Therefore, less energy will be absorbed by the salt and the phase transition of the salt may not occur.

In this framework, some embodiments include a technique based on additive manufacturing that is able to produce porous layers with precise control on porosity and pore size distribution, with parameters that can be tuned to match criteria set by the application. Moreover, a gradient in porosity can be implemented to have a material with higher density (and higher concentration of catalytic sites) and low porosity towards a membrane, where the size of the bubbles is small, and with a porosity and pore size that increase with the thickness of the porous layer, matching the growing of the bubbles during the reaction.

### Detailed Objectives and Development Stages

From the fundamental perspective it is desired to understand how to control manufacturing parameters to arrive at porous media morphology with application to heat transfer and energy-conversion technologies with two-phase flow. A broad impact is to generate an understanding of the level of control for SLM and of how gradient in morphological properties, such as porosity and particle sizes, can affect transport properties. To gain this understanding, three objectives are put forward:
Objective 1: Determine the degree of control that can be achieved with sacrificial materials for SLM and by varied laser power, hatch size, powder size to achieve controlled morphology of porous media.
Objective 2: Understand the morphological properties of materials manufactured in Objective 1 and their applicability for effective transport for energy technologies.
Objective 3: Leverage knowledge from Objectives 1 and 2 to identify nano- and micro-scale morphologies for energy applications, with the help of machine learning generate optimal topologies for heat transfer and electrolysis.
   Objective 1. Understand the Fundamentals of Morphology Control of the Powder SLM Manufacturing Process with Laser Volume Density and Sacrificial Pore-Formers.

### Pore Size and Porosity Controls by Using Sacrificial Materials

As previously discussed, in certain embodiments, sacrificial template can be employed to overcome the constraints of SLM in determining the porosity of media.

In a first stage in certain embodiments, development is made of control over the process, optimizing parameters involved towards tunable characteristics to the maximum extent.

An extensive sensitivity evaluation can be carried out to determine the effect of several operating conditions and material composition on the parameters of a porous layer:
Geometry: the input geometry in SLM, that can change from solid, bulk to open 3D meshes of various geometries.
Laser Fluence: represents the energy given to the material for the phase transition—it can be tuned to be enough to melt the metal but not the salt. A set of fabrications for the calibration can be performed.
Spot size: it is one of the factors that determine the resolution that can be achieved in the drawing of a mesh.
Laser speed: it drives, together with the laser power, the energy load transferred to the material and influences the phase transition process.

Other parameters also can be considered in certain embodiments, but related to the nature of the mixture of a metal and a salt:
Particle dimensions: the dimension of the salt particles will eventually determine the pore size and pore size distribution in the final part.
Material: different metals have different thermodynamic parameters so different operating conditions should be adopted for each metal/salt mixture. Moreover, each material interacts differently with the laser and the thermal management involved in the interaction can determine the resolution (e.g., if a metal has high thermal conductivity, the heat will be transferred quickly to the particles nearby, leaving less energy for the phase transformation).
Metal-to-salt ratio: it will determine the porosity of the medium as well as creating preferential percolation pathways that will make the etching process easier.
Absorption spectrum of the pore former: it can modify the energy absorbed by the sacrificial pore former.

Once the effect of each parameter is identified, the fabrication process can be performed to tune a material tailoring to desired applications.

Geometric Parameters: Porosity, Pore Size Distribution, Tortuosity, Surface Area X-Ray tomography

To evaluate a porous medium it is desired to individually analyze several figures of merit that are instrumental to a possible application. Those figure of merit are the following:
Porosity: the void fraction of an overall volume
Pore size distribution
Tortuosity: as an indication of the complexity of a pore network
Surface area: specific either to the mass or the surface (m²*g⁻¹, m²*m⁻²) is useful when dealing with heat and mass transfer, especially in catalysis

Nitrogen adsorption methods can be used in certain embodiments to quantify porous media: they exploit the physisorption of nitrogen on a porous surface in cryogenic conditions. From the analysis of the absorption isotherms it is possible to calculate through different models, both in terms of the surface area (Brunauer-Emmett-Teller (BET)) and pore size distribution.

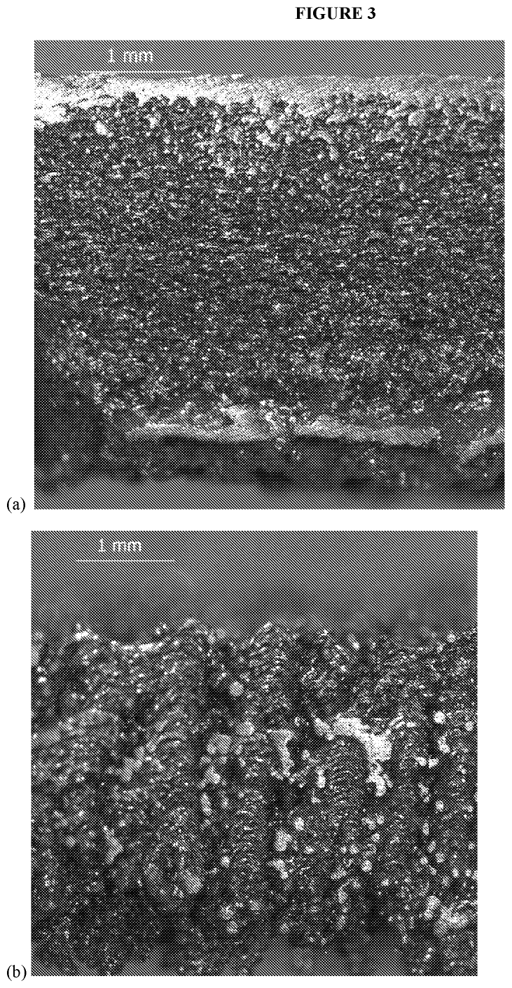

Microscopic images, energy-dispersive X-ray spectroscopic images, and X-ray diffraction data can be used in certain embodiments to quantify morphological details or chemical compositions. For example, in FIG. 2, the SLM of a specific embodied stainless-steel part is shown.

### Broader Impacts

Some embodiments of the disclosed can benefit the design of porous materials for energy applications such as heat pipes or electrolyzers.

Heat pipes. Evaporative cooling continues to be one of the most promising approaches to meet future thermal management demands. The theoretical kinetic limit of 5-20 kW/cm² has not been realized because it is challenging to sustain an evaporating thin film on surfaces supported by continuous liquid transport. Conflicts between the coupled hydraulics and thermodynamics of an evaporating thin film can be addressed through the development of improved porous media that are designed to simultaneously increase both the rate of fluid delivery and the rate of liquid-vapor heat transfer. The advantage of heterogeneous porous media is the ability to spatially tune the properties to locally modify the thermofluidic parameters based on the dominant transport mechanisms. For example, conductive heat spreading (e.g., thermal conductivity k) is the dominant mode of heat dissipation at the location of the heat source, whereas fluid delivery (e.g., permeability K and capillary pressure ΔP) is the dominant transport mechanism far from the heat source.

Electrolyzer. Concepts for a green economy are heavily relying on hydrogen as the energy vector; therefore, efficient production is desired to achieve ambitious long-term goals. Electrolysis is an efficient way to produce hydrogen that is at the same time compatible with renewable energy: a decrease in the specific cost of electrolyzers and of hydrogen production could effectively lead to a transition to an effective decarbonization of the entire energy system. Moreover, hydrogen production is a viable power outlet to store surplus renewable energy that cannot be injected in the grid, increasing the utilization factor of green sources. Advances in electrolyzers bring the technology towards high performing devices with high efficiencies, but the nature of reactions involved still imposes a constraint to the rate of production. In particular, an anodic side of a proton exchange electrolyzer, where oxygen is produced, is subject to harsh conditions of potential and pH, and therefore special titanium-based porous layers can be employed to avoid the fast corrosion that the electrode is facing. Moreover, due to the heavy production of oxygen bubbles, the catalytic sites can be locally isolated from the reaction because of the lack of liquid water as a reactant. An optimal porous structure is desired to manage the turnover between reactants and products in the electrode, maximizing the reaction rate.

### EXAMPLES

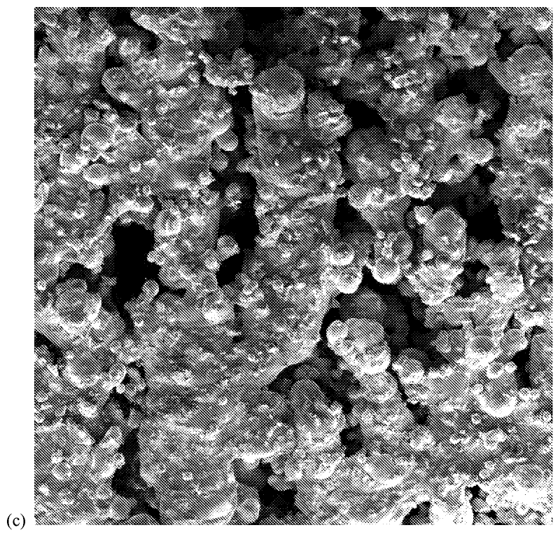

A SLM machine (SLM125HL, SLM Solutions Gmbh., Germany) was loaded with a mixture of stainless steel 316L powder (average particle size 40-65 μm) and silica gel spheres (average particle size 46-65 μm). The laser employed has a wavelength of 1040 nm. The mixture is prepared with 50% in volume of metal and 50% silica. The two powders were previously mixed using a ball milling equipment. Samples of 1 cm×1 cm×4 mm were printed in the SLM for imaging purpose. Different laser powers were investigated in the range of 50-400 W, with an optimal result around 100 W. After the print, the samples were cut using a diamond saw and the cross section was polished using a grinder/polisher. The resulting composition is shown in FIGS. 3(b) and 3(c). The same printing was performed using pure stainless steel 316L for comparison (see FIG. 3(a)).

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, a first numerical value can be “substantially” or “about” the same as or equal to a second numerical value if the first numerical value is within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

1. A manufacturing method comprising:
(1a) forming a layer, including: depositing a starting material including a mixture of a metal and a sacrificial material; and applying a laser beam to the deposited starting material to consolidate the deposited starting material and form the layer;
(1b) optionally repeating (1a) one or more times; and
(1c) at least partially removing the sacrificial material to form a porous metal part.

2. The manufacturing method according to claim 1, wherein depositing the starting material includes depositing the starting material as a powder.

3. The manufacturing method according to claim 2, wherein the starting material is a mixture of a power of the metal and a powder of the sacrificial material.

4. The manufacturing method according to claim 3, wherein the powder of the sacrificial material includes particles having an average size in a range of about 1 nm to about 70 μm, about 1 nm to about 50 μm, about 1 nm to about 10 μm, about 1 nm to about 1 μm, about 1 nm to about 800 nm, or about 1 nm to about 500 nm.

5. The manufacturing method according to claim 3, wherein the powder of the sacrificial material includes particles having a size distribution that is monodisperse.

6. The manufacturing method according to claim 3, wherein the powder of the sacrificial material includes particles having a size distribution that is polydisperse.

7. The manufacturing method according to claim 3, wherein the resulting porous metal part includes pores having an average size and a size distribution corresponding to an average size and a size distribution of particles of the sacrificial material.

8. The manufacturing method according to claim 3, wherein the resulting porous metal part has a porosity corresponding to a ratio of the powder of the sacrificial material and the power of the metal.

9. The manufacturing method according to claim 1, wherein applying the laser beam to the deposited starting material is according to a mesh pattern.

10. The manufacturing method according to claim 9, wherein the resulting porous metal part includes additional pores having an average size and a size distribution corresponding to the mesh pattern.

11. The manufacturing method according to claim 1, wherein the sacrificial material remains in a liquid or solid state while applying the laser beam to the deposited starting material.

12. The manufacturing method according to claim 1, wherein the starting material is an ionic salt.

13. The manufacturing method according to claim 1, wherein removing the sacrificial material includes dissolving the sacrificial material in a solvent.

14. The manufacturing method according to claim 13, wherein dissolving the sacrificial material is performed at an elevated temperature.

15. A manufacturing method comprising:
(2a) forming a first layer, including: depositing a first starting material including a first mixture of a metal and a sacrificial material; and applying a laser beam to the deposited first starting material to consolidate the deposited first starting material and form the first layer;
(2b) optionally repeating (2a) one or more times;
(2c) forming a second layer on the first layer, including: depositing a second starting material including a second mixture of the metal and the sacrificial material; and applying a laser beam to the deposited second starting material to consolidate the deposited second starting material and form the second layer;
(2d) optionally repeating (2c) one or more times; and
(2e) at least partially removing the sacrificial material to form a porous metal part.

16. The manufacturing method according to claim 15, wherein depositing the first starting material includes depositing the first starting material as the first mixture of a power of the metal and a powder of the sacrificial material, and depositing the second starting material includes depositing the second starting material as the second mixture of a power of the metal and a powder of the sacrificial material.

17. The manufacturing method according to claim 16, wherein an average size or a size distribution of particles of the sacrificial material in the first mixture is different than an average size or a size distribution of particles of the sacrificial material in the second mixture.

18. The manufacturing method according to claim 16, wherein a ratio of the powder of the sacrificial material and the power of the metal in the first mixture is different than a ratio of the powder of the sacrificial material and the power of the metal in the second mixture.

19. The manufacturing method according to claim 15, wherein applying the laser beam to the deposited first starting material is according to a first mesh pattern.

20. The manufacturing method according to claim 19, wherein the resulting porous metal part includes additional pores having an average size and a size distribution corresponding to the first mesh pattern.

21. The manufacturing method according to claim 20, wherein applying the laser beam to the deposited second starting material is according to a second mesh pattern.

22. The manufacturing method according to claim 21, wherein the resulting porous metal part includes additional pores having an average size and a size distribution corresponding to the second mesh pattern.

23. The manufacturing method according to claim 22, wherein the first mesh pattern is different than the second mesh pattern.

24. A porous metal part formed by the manufacturing methods of claim 1.

25. The porous metal part of claim 24, which is a catalyst support.

26. The porous metal part of claim 24, which is a porous transport layer of a hydrogen generator.

27. The porous metal part of claim 24, which is a porous medium of a heat pipe.

28. The porous metal part of claim 24, which is a component of an implantable device.
